SEQUENCE LISTING (6 sequences)

SEQ ID NO: 1 - protein, 241 aa, Trichoderma reesei
PVNSSLPLRRIIPRSFSSIAMAPASQVVSALMLPALALGAAIQPRGADIVGGTAASLGEFPYIVSLQNPNQGGHFCGGVLVNANTVVTAAHCSVVYPASQIRVRAGTLTWNSGGTLVGVSQIIVNPSYNDRTTDFDVAVWHLSSPIRESSTIGYATLPAQGSDPVAGSTVTTAGWGTTSENSNSIPSRLNKVSVPVVARSTCQADYRSQGLSVTNNMFCAGLTQGGKDSCSGDSGGPIVDA

SEQ ID NO: 2 - protein, 254 aa, Trichoderma harzianum
MAPVSQIVSALMLPALALGAAIEPRGADIVGGTAAALGEFPYIVSLSTQGSHFCGGVLVNANTVITAAHCSVDFSASQVKVRAGTLTWASGGTQVGVSRIIVNPSYNSRTTDFDVAVWKLASSIPESSTIGYATLPAAGSDPAAGSIVTTAGWGTTSEGSSSLPARLNKVSVPVVSRATCQSEYGRSAVTTNMFCAAQAQGGKDSCQGDSGGPIVDANGVLQGLVSWGNGCAEAGFAGVYSRLGSLLSFVQANL

SEQ ID NO: 3 - protein, 264 aa, Pyrenophora tritici-repentis
MRFQTIIAFALPALALAVPTPQDPNFEFPADTPDDDIVGGSTAASGEFPYIVSLQVGGSHICGGSLINGNTVVTAAHCSVSSVIGSVSNLRVRAGSLSRSSGGTLVGVSQVIVNPNYRSSSQDYDIAIWKLSSSIPTSSTIGYVGLPASGSDPAAGATVTVAGWGTLSSGGSSPNALYKVSVPVVSRTSCRSSYGSSSITNNMVCAGLTNGGKDSCQGDSGGPLVDASKTLVGVVSFGQGCALPGYPGVYSRVSTLLSFIQQYD

SEQ ID NO: 4 - protein, 258 aa, Trichoderma harzianum
MAPVLAIASVLAALPALTMGAAITPRGSDIVGGTTAALGEFPYIVSLSTGGSHFCGGVLIDSRTVVTAGHCTIDQRASSVKVRAGTLTWASGGTQVGVSSLTLHPSYTVDSQGVPDNDVGVWHLATAIPTSSTIGYATLPASGSDPAAGTTLTVAGWGTTSENSNSLPSTLRKVSVPVVARATCDSDYDGEISNNMFCAAVAAGGKDSCSGDSGGPIIDPSGTLVGVVSWGQGCAERGFPGVYTRLGNYVSFINSNRG

SEQ ID NO: 5 - protein, 289 aa, Fusarium acuminatum
ALTTQSGAPWGLGAISHKSSGSTSYIYDTTAGSGSYGYVVDSGINIAHTDFGGRATLGYNAAGGAHTDTLGHGTHVAGTIGGTKYGVSKKANLISVKVFAGNQAATSVILDGFNWAVNDITSKGRAGKSVINMSLGGPSSATWTTAINAGYNAGVLSVVAAGNGDVNGNPLPVSSQSPANAPNALTVAAIDSNWRTASFTNYGAGVDIFGPGVNILSAWIGSSTATNTISGTSMASPHLAGLALYLQVLEGLSTPAAVTNRIKALGTSGKVTGSLSGSPNLVAYNGNGA

SEQ ID NO: 6 - protein, 289 aa, Fusarium equiseti
ALTTQSNAPWGLAAISRRTPGGSTYTYDTTAGAGTYGYVVDSGINTAHTDFGGRASLGYNAAGGAHTDTLGHGTHVAGTIASNTYGVAKRANVISVKVFVGNQASTSVILAGFNWAVNDITSKNRASRSVINMSLGGPSSQTWATAINAAYSQGVLSVVAAGNGDSNGRPLPASGQSPANVPNAITVAAADSSWRTASFTNYGPEVDVFGPGVNIQSTWYTSNSATNTISGTSMACPHVAGLALYLQALENLNTPAAVTNRIKSLATTGRITGSLSGSPNAMAFNGATA

(19) United States
(12) Patent Application Publication
(10) Pub. No.: US 2015/0353870 A1
(43) Pub. Date: Dec. 10, 2015

(54) CLEANING AND/OR TREATMENT COMPOSITIONS

(71) Applicant: The Procter & Gamble Company, Cincinnati, OH (US)

(72) Inventor: Neil Joseph LANT, Newcastle upon Tyne (GB)

(21) Appl. No.: 14/833,516

(22) Filed: Aug. 24, 2015

Related U.S. Application Data
(63) Continuation of application No. 13/278,201, filed on Oct. 21, 2011, now abandoned.
(60) Provisional application No. 61/414,650, filed on Nov. 17, 2010.
(60) Provisional application No. 61/408,070, filed on Oct. 29, 2010.

(30) Foreign Application Priority Data
Oct. 29, 2010 (US) PCT/US2010/054670
Dec. 17, 2010 (US) PCT/US2010/057029

(51) Int. Cl. C11D 3/386; C12N 9/58

(52) U.S. Cl. CPC C11D 3/386; C12N 9/58; C12Y 304/21

(57) ABSTRACT

This invention relates to compositions comprising certain fungal serine proteases and processes for making and using such compositions including the use of such compositions to clean and/or treat a situs.

### FIELD OF INVENTION

This invention relates to cleaning and/or treatment products comprising fungal serine proteases as well as methods of making and using such cleaning and/or treatment products.

### BACKGROUND OF THE INVENTION

Detergent manufacturers incorporate proteases into their products to provide good cleaning of proteinaceous stains (such as blood). However, given the sustainability and consumer trends to lower wash temperatures it is proving increasingly difficult to deliver such consumer acceptable benefits at lower wash temperatures as current proteases have very low activity levels, for example 10% of their maximum activity, in the typical low wash temperatures of 5° C. to 20° C. Thus, there remains a need to improve the cleaning and freshness profile of consumer products that will be used at low wash temperatures. Applicant has surprisingly recognized that when consumer products are formulated with certain fungal proteases, 50% to 70% of the enzyme's maximum activity is obtained. Thus, cleaning of proteinaceous stains is greatly improved and surprisingly the performance of other cleaning ingredients is enhanced. For example, the performance of proteases other than the aforementioned fungal protease is improved, lipolytic action of lipases is enhanced, amylolytic action of amylases is enhanced, the catalytic bleaching action of bleach catalysts is increased, hueing agent performance is increased, the action of chelants is enhanced and the performance of perfume microcapsules is improved.

### SUMMARY OF THE INVENTION

This invention relates to cleaning and/or treatment products comprising fungal proteases and processes for making and using such products. Such compositions provide improved cleaning and freshness. Such proteases are wild types or are derived from such wild types, by substitution, insertion and/or deletion of one or more of the parent enzymes' amino acids.

### DETAILED DESCRIPTION OF THE INVENTION

### Definitions

As used herein, the term “cleaning and/or treatment composition” includes, unless otherwise indicated, granular or powder-form all-purpose or “heavy-duty” washing agents, especially laundry detergents; liquid, gel or paste-form all-purpose washing agents, especially the so-called heavy-duty liquid types; liquid fine-fabric detergents; hand dishwashing agents or light duty dishwashing agents, especially those of the high-foaming type; machine dishwashing agents, including the various tablet, unit dose liquid tablets/pouches, impregnated nonwoven sheets, granular, liquid and rinse-aid types for household and institutional use; liquid cleaning and disinfecting agents, including antibacterial hand-wash types, laundry bars, car or carpet shampoos, bathroom cleaners; hair; as well as cleaning auxiliaries such as bleach additives and “stain-stick” or pre-treat types.

As used herein, the phrase “is independently selected from the group consisting of . . . ” means that moieties or elements that are selected from the referenced Markush group can be the same, can be different or any mixture of elements.

As used herein, articles, for example, “a” and “an” when used in a claim, are understood to mean one or more of what is claimed or described.

As used herein, the terms “include”, “includes” and “including” are meant to be non-limiting.

Unless otherwise noted, all component or composition levels are in reference to the active level of that component or composition, and are exclusive of impurities, for example, residual solvents or by-products, which may be present in commercially available sources.

Unless otherwise noted, the enzymes of the present invention are expressed in terms of active protein level and are exclusive of impurities, for example, residual solvents or by-products, which may be present in commercially available sources.

The term “identity” in the context of two polypeptide sequences refers to the residues in the two sequences that are the same when aligned for maximum correspondence, as measured using one of the following sequence comparison or analysis algorithms. The term “optimal alignment” refers to the alignment giving the highest percent identity score. “Percent sequence identity,” “percent amino acid sequence identity,” with respect to two amino acid sequences, refer to the percentage of residues that are identical in the two sequences when the sequences are optimally aligned. Thus, 80% amino acid sequence identity means that 80% of the amino acids in two optimally aligned polypeptide sequences are identical. Alignment of the two polypeptide sequences may be conducted using the programs or algorithms (e.g., BLAST, ALIGN, CLUSTAL) using standard parameters.

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Suitable Fungal Serine Proteases

In one aspect the composition comprises a fungal serine protease having at least 56%, 70%, 75%, 80%, 85%, 90%, 95%, 99%, or even complete identity to SEQ ID NO: 1. SEQ ID NO: 1 is the amino acid sequence of a fungal serine protease derived from Trichoderma reesei strain QM9414. In one aspect, the fungal serine protease is the endopeptidase from Trichoderma harzianum strain CAL25577 having the amino acid sequence of SEQ ID NO: 2, which has 81% identity to SEQ ID NO: 1. In another aspect, the fungal serine protease is the trypsin precursor from Pyrenophora tritici-repentis strain Pt-1c-BFP having the amino acid sequence of SEQ ID NO: 3, which has 63% identity to SEQ ID NO: 1. In another aspect, the fungal serine protease is the trypsin-like protease from Trichoderma harzianum strain CECT 2413 having the amino acid sequence of SEQ ID NO: 4, which has 65% identity to SEQ ID NO: 1.

In another aspect the composition comprises a fungal serine protease having at least 81%, 85%, 90%, 95%, 99%, or even complete identity to SEQ ID NO: 5. SEQ ID NO: 5 is the amino acid sequence of a fungal serine protease derived from Fusarium acuminatum strain CBS 124084.

In another aspect the composition comprises a fungal serine protease having at least 86%, 90%, 95%, 99%, or even complete identity to SEQ ID NO: 6. SEQ ID NO: 6 is the amino acid sequence of a fungal serine protease derived from Fusarium equiseti strain CBS 119568.

The fungal serine proteases can be produced using standard biochemical means. For example, a procedure for the isolation of the fungal serine protease derived from Trichoderma reesei strain QM9414 defined by SEQ ID NO: 1, is found in Example 1 of this specification. In other aspects, the fungal serine protease is a protein engineered variant of one of the four wild-type enzymes defined by SEQ ID NOS: 1-4, having at an amino acid sequence with at least 56% identity to SEQ ID NO: 1. In other aspects, the fungal serine protease is a protein engineered variant of one of the two wild type enzymes defined by SEQ ID NOS 5-6, having at least 81% identity to SEQ ID NO: 5 or at least 86% identity to SEQ ID NO: 6.

Protein engineered variants can be produced using standard procedures well-known to those skilled in the art. Multiple amino acid substitutions can be made and tested using known methods of mutagenesis, recombination and/or shuffling followed by a relevant screening procedure. Briefly, these methods involve simultaneously randomizing two or more positions in a polypeptide, or recombination/shuffling of different mutations followed by selecting a polypeptide for functionality, and then sequencing the mutagenized polypeptides to determine the spectrum of allowable substitutions at each position. Other methods that can be used include phage display and region-directed mutagenesis. Mutagenesis/shuffling methods as disclosed above can be combined with high-throughput, automated screening methods to detect activity of cloned, mutagenized polypeptides in host cells. Mutagenized DNA molecules that encode active polypeptides can be recovered from the host cells and rapidly sequenced using modem equipment. These methods allow the rapid determination of the importance of individual amino acid residues in a polypeptide of interest, and can be applied to polypeptides of unknown structure. Using the methods discussed above, one of ordinary skill in the art can identify and/or prepare a variety of polypeptides that are substantially homologous to the polypeptides of SEQ ID NOS: 1-4 above and retain the proteolytic activity of the wild-type protein, as detected, for example using the artificial substrate azo-casein. When producing such variants, the catalytic active site residues should be preserved, i.e. His-91, Asp-136 and Ser-234 for SEQ ID 1, although substitutions, insertions and deletions to the other regions of the polypeptide chain may be beneficial in enhancing performance of the enzyme in a cleaning and/or treatment composition. Examples of such changes are substitutions to surface residues in order to change the charge of the enzyme and hence influence its deposition onto surfaces such as textiles, skin or hard surfaces. Other changes may be beneficial in reducing the sensitivity of the enzyme to autolysis, or attack by other proteases, for example by substituting sites that are susceptible to proteolytic attack. Other changes may be beneficial in reducing the sensitivity of the enzyme to denaturation by temperature, surfactant, chelating agent or bleaching agents.

Cleaning and/or Treatment Compositions

In one aspect, a cleaning and/or treatment composition comprising a fungal serine protease selected from the group consisting of a fungal serine protease having:
a) SEQ ID NO: 1;
b) SEQ ID NO: 5;
c) SEQ ID NO: 6;
d) a fungal serine protease having at least 56%, 70%, 75%, 80%, 85%, 90%, 95%, 99%, identity to SEQ ID NO: 1; and
e) a fungal serine protease having at least 81%, 85%, 90%, 95%, 99%, identity to SEQ ID NO: 5;
f) a fungal serine protease having at least 86%, 90%, 95%, 99%, identity to SEQ ID NO: 6;
g) combinations there of; and
   an additional cleaning material is disclosed.

In one aspect, said cleaning and/or treatment composition comprises, based on total composition weight, from about 0.00001% to about 2%, from about 0.0001% to about 1%, from about 0.0005% to about 1%, from about 0.001% to about 0.5% or even from about 0.002% to about 0.25% of said fungal serine protease.

In one aspect of said cleaning and/or treatment composition, said a fungal serine protease has at least 56%, 70%, 75%, 80%, 85%, 90%, 95%, 99%, identity to SEQ ID NO: 1 is selected from the group consisting of fungal serine proteases having SEQ ID NO: 2, SEQ ID NO: 3; SEQ ID NO: 4 and mixtures there of.

In one aspect of said cleaning and/or treatment composition, said additional cleaning material is being selected from the group consisting of surfactants, chelating agents, dye transfer inhibiting agents, dispersants, additional enzymes, and enzyme stabilizers, catalytic materials, bleaching agents, polymeric dispersing agents, clay soil removal/anti-redeposition agents, brighteners, suds suppressors, dyes, perfumes, perfume microcapsules, structure elasticizing agents, fabric softeners, carriers, hydrotropes, processing aids, solvents, pigments, hueing agents, photobleaches, structurants, and mixtures thereof.

In one aspect of said cleaning and/or treatment composition, said cleaning and/or treatment composition comprises an additional enzyme.

In one aspect of said cleaning and/or treatment composition, said additional enzyme is selected from the group consisting of hemicellulases, peroxidases, proteases, cellulases, xylanases, lipases, phospholipases, perhydrolases, esterases, cutinases, pectinases, mannanases, pectate lyases, keratinases, reductases, oxidoreductases, phenoloxidases, lipoxygenases, ligninases, pullulanases, tannases, pentosanases, glucanases, arabinosidases, hyaluronidase, chondroitinase, laccase, amylases, and mixtures thereof.

In one aspect of said cleaning and/or treatment composition, said additional enzyme is selected from the group consisting of: first cycle lipases; cutinases; alpha-amylases; bacterial proteases; microbial-derived endoglucanases; and mixtures thereof.

In one aspect of said cleaning and/or treatment composition, said cleaning and/or treatment composition comprises a surfactant, selected from the group of: anionic surfactants selected from the group consisting of linear alkylbenzene-sulfonate (LAS), alcohol ethoxysulfate (AES), mid-branched alkyl sulfates (HSAS) and mixtures thereof; non ionic alcohol ethoxylates, amine oxides; and mixtures thereof.

In one aspect of said cleaning and/or treatment composition, said cleaning and/or treatment composition comprising a polymer, selected from the group consisting of polyacrylates; maleic/acrylic acid copolymers; cellulose-derived polymers; polyethyleneimine polymer; and mixtures thereof.

In one aspect of said cleaning and/or treatment composition, said cleaning and/or treatment composition comprises a fabric hueing agent selected from the group consisting of dyes; dye-clay conjugates comprising at least one cationic/basic dye and a smectite clay; and mixtures thereof.

In one aspect of said cleaning and/or treatment composition, said cleaning and/or treatment composition comprises, based on total product weight, from about 0.00003% to about 0.3% hueing agent.

In one aspect of said cleaning and/or treatment composition, said cleaning and/or treatment composition comprises, based on total product weight, less than 15% builder.

In one aspect of said cleaning and/or treatment composition, said cleaning and/or treatment composition is a multi-compartment unit dose.

In one aspect of said cleaning and/or treatment composition, said cleaning and/or treatment composition is in the form of a multi-compartment unit dose, wherein the fungal serine protease is in a different compartment to any additional enzymes and/or chelant.

In one aspect of said cleaning and/or treatment composition, said cleaning and/or treatment composition is a hand dishwashing or machine dishwashing composition.

In one aspect, said cleaning and/or treatment composition comprises, based on total cleaning and/or treatment composition weight, a total of no more than 20% water, a total of no more than 15% water or even a total of no more than 10% water.

In one aspect, said cleaning and/or treatment composition comprises based on total cleaning and/or treatment composition weight, from about 10% to about 70%, or even from about 20% to about 60% of a water-miscible organic solvent, said water-miscible organic solvent in one aspect having a molecular weight of greater than 70 Daltons, in one aspect said water-miscible organic solvent in one aspect having a molecular weight of greater than 70 Daltons to about 1000 Daltons.

In one aspect, said cleaning and/or treatment composition comprising a perfume microcapsule comprising a core and a shell that encapsulates said core, said perfume microcapsule having a D[4,3] average particle of from about 0.01 microns to about 200 microns.

In one aspect, of the cleaning and/or treatment composition said composition may comprise
a) a first wash lipase selected from the group consisting variants of the Humicola lanuginosa lipase comprising a substitution of an electrically neutral or negatively charged amino acid with R or K at any of positions 3, 224, 229, 231 and 233, in one aspect, a variant comprising T231R and N233R mutations;
b) a hueing dye selected from the group consisting of direct violet 7, direct violet 9, direct violet 11, direct violet 26, direct violet 31, direct violet 35, direct violet 40, direct violet 41, direct violet 51, direct violet 66, direct violet 99, acid violet 50, acid blue 9, acid violet 17, acid black 1, acid red 17, acid blue 29, solvent violet 13, disperse violet 27 disperse violet 26, disperse violet 28, disperse violet 63 and disperse violet 77, basic blue 16, basic blue 65, basic blue 66, basic blue 67, basic blue 71, basic blue 159, basic violet 19, basic violet 35, basic violet 38, basic violet 48; basic blue 3, basic blue 75, basic blue 95, basic blue 122, basic blue 124, basic blue 141, thiazolium dyes, reactive blue 19, reactive blue 163, reactive blue 182, reactive blue 96, and polymeric dyes;
c) a bacterial protease selected from the group consisting of wild-type and variants of subtilisins derived from Bacillus lentus, B. alkalophilus, B. subtilis, and B. amyloliquefaciens.
d) a bacterial amylase selected from the group consisting of wild-type and variants of amylase AA560 from Bacillus sp. DSM 12649, and wild-type and variants of amylase SP722 from Bacillus sp. NCIB 12513.
e) an endo-beta-1,4-glucanase selected from the group consisting of wild-type and variants of the 20 kDa endoglucanase from Melanocarpus albomyces, wild-type and variants of the endoglucanase from Bacillus sp. AA349; and wild-type and variants of the XYG1006 endoglucanase from Paenibacillus polymyxa
f) a perhydrolase selected from the group consisting of variants of the Mycobacterium smegmatis perhydrolase, and variants of the CE-7 perhydrolases;
g) a perfume microcapsule selected from the group consisting of core/shell perfume microcapsules, in one aspect comprising a melamine/formaldehyde resin shell;
h) a carboxymethylcellulose is selected from the group consisting of carboxymethycellulose derivatives having a degree of carboxymethyl substitution of from about 0.5 to about 0.95; and
i) a bleaching material selected from the group consisting of catalytic metal complexes, photobleaches, bleach activators, hydrogen peroxide, sources of hydrogen peroxide, pre-formed peracids, bleach boosters and mixtures thereof.

In one aspect, the aforementioned additional enzyme may be selected from the group consisting of: lipases, including “first cycle lipases” derived from the Humicola lanuginosa lipase described in U.S. Pat. No. 6,939,702 B1, a variant of SEQ ID No. 1, in U.S. Pat. No. 6,939,702 B1 having at least 90% identity to SEQ ID No. 1 comprising a substitution of an electrically neutral or negatively charged amino acid with R or K at any of positions 3, 224, 229, 231 and 233, or even a variant comprising T231R and N233R mutations, such variant being sold under the tradename Lipex®; cutinases defined by E.C. Class 3.1.1.73, preferably displaying at least 90%, or 95%, or most preferably at least 98% identity with a wild-type derived from one of Fusarium solani, Pseudomonas mendocina or Humicola insolens; alpha-amylases, including amylase AA560 from Bacillus sp. DSM 12649, and wild-type and variants of amylase SP722 from Bacillus sp. NCIB 12513, with examples Natalase® (Novozymes), Stainzyme® (Novozymes), and Stainzyme Plus (Novozymes); serine proteases, including neutral or alkaline microbial serine proteases, such as subtilisins (EC 3.4.21.62), including those derived from Bacillus lentus, B. alkalophilus, B. subtilis, B. amyloliquefaciens described in U.S. Pat. No. 6,312,936 B1, U.S. Pat. No. 5,679,630, U.S. Pat. No. 4,760,025, with examples Alcalase® (Novozymes), FNA (Genencor), Savinase® (Novozymes), Purafect (Genencor), KAP (Kao), Everlase™ (Novozymes), Purafect OxP™ (Genencor), FN4 (Genencor), BLAP S (Henkel), BLAP X (Henkel), Esperase® (Novozymes), Kannase™ (Novozymes) and Properase™ (Genencor); microbial-derived endoglucanases exhibiting endo-beta-1,4-glucanase activity (E.C. 3.2.1.4), including a bacterial polypeptide endogenous to a member of the genus Bacillus which has a sequence of at least 90%, 94%, 97% and even 99% identity to SEQ ID NO:2 in US 2005/0112749 A1—such an enzyme being commercially available under the tradename Celluclean™ by Novozymes A/S, and mixtures thereof; oxidoreductases, for example oxidases such as glucose, choline or carbohydrate oxidases, oxygenases, catalases, peroxidases, like halo-, chloro-, bromo-, lignin-, glucose- or manganese-peroxidases, dioxygenases or laccases (phenoloxidases, polyphenoloxidases). Suitable commercial products are sold under the Guardzyme® and Denilite® ranges from Novozymes. In one aspect, organic, for example, aromatic compounds are incorporated with the bleaching enzyme. While not being bound by theory, it is believed that these compounds interact with the bleaching enzyme to enhance the activity of the oxidoreductase (enhancer) or to facilitate the electron flow (mediator) between the oxidizing enzyme and the stain typically over strongly different redox potentials; perhydrolases which catalyse the formation of peracids from an ester substrate and peroxygen source. Suitable perhydrolases include variants of the Mycobacterium smegmatis perhydrolase, variants of so-called CE-7 perhydrolases, and variants of wild-type subtilisin Carlsberg possessing perhydrolase activity.

Any of the aspects of the cleaning and/or treatment compositions described in the present specification may comprise a surfactant, including a surfactant selected from the group of anionic surfactants including anionic surfactants selected from the group consisting of linear alkylbenzene-sulfonate (LAS), alcohol ethoxysulfate (AES), mid-branched alkyl sulfates (HSAS) and mixtures thereof; non-ionic surfactants including alcohol ethoxylates, for example alcohol ethoxylates having a chain length of from 1 to 14 carbons, or 12 to 14 carbons; amine oxides and mixtures thereof.

Any of the aspects of the cleaning and/or treatment compositions described in the present specification may comprise a polymer, including polymers selected from the group consisting of polyacrylates, maleic/acrylic acid copolymers, cellulose-derived polymers, including carboxymethylcellulose and methyl hydroxyethylcellulose, polyethyleneimine polymers and mixtures thereof. In one aspect, carboxymethylcellulose is selected from the group consisting of carboxymethycellulose derivatives having a degree of carboxymethyl substitution of from about 0.5 to about 0.95

Any of the aspects of said cleaning and/or treatment compositions described in the present specification may comprise a builder selected from the group consisting of citric acid, C₁₂-C₁₈ fatty acid, aluminosilicates, including zeolites A, X and/or Y, sodium tripolyphosphate and mixtures thereof.

Any of the aspects of the cleaning and/or treatment compositions described in the present specification may comprise a fabric hueing agent selected from the group consisting of direct violet 7, direct violet 9, direct violet 11, direct violet 26, direct violet 31, direct violet 35, direct violet 40, direct violet 41, direct violet 51, direct violet 66, direct violet 99, acid violet 50, acid blue 9, acid violet 17, acid black 1, acid red 17, acid blue 29, solvent violet 13, disperse violet 27 disperse violet 26, disperse violet 28, disperse violet 63 and disperse violet 77, basic blue 16, basic blue 65, basic blue 66, basic blue 67, basic blue 71, basic blue 159, basic violet 19, basic violet 35, basic violet 38, basic violet 48; basic blue 3, basic blue 75, basic blue 95, basic blue 122, basic blue 124, basic blue 141, thiazolium dyes, reactive blue 19, reactive blue 163, reactive blue 182, reactive blue 96, polymeric dyes such as Liquitint® Violet DD (Milliken), Liquitint® Violet CT (Milliken, Spartanburg, USA) and Azo-CM-Cellulose (Megazyme, Bray, Republic of Ireland). Other suitable hueing agents are hueing dye-photobleach conjugates, such as the conjugate of sulphonated zinc phthalocyanine with direct violet 99. A particularly preferred hueing agent is a combination of acid red 52 and acid blue 80, or the combination of direct violet 9 and solvent violet 13, dye-clay conjugates comprising at least one cationic/basic dye and a smectite clay and mixtures thereof.

Any of the aspects of the cleaning and/or treatment compositions described in the present specification may comprise, based on total product weight, from about 0% to about 3%, from about 0.0001% to about 0.5%, or even from about 0.0005% to about 0.3% photobleach and/or from about 0.00003% to about 0.3%, from about 0.00008% to about 0.05%, or even from about 0.0001% to about 0.04% hueing agent.

It is understood that any of the aspects of the cleaning and/or treatment compositions described in the present specification may comprise any combinations of materials and parameters disclosed herein. Thus, the cleaning and/or treatment compositions described in the present specification may comprise multiple materials, for example, enzymes, surfactants, polymers builders and fabric hueing agents.

Enzymes suitable for use in the present cleaning and/or treatment compositions can be obtained from Genencor International, Palo Alto, Calif., U.S.A; Novozymes A/S, Bagsvaerd, Denmark; Sigma-Aldrich Company Ltd, Dorset, UK; and AB Enzymes, Darmstadt, Germany.

Surfactants suitable for use in the present cleaning and/or treatment compositions can be obtained from Stepan, Northfield, Ill., USA; Huntsman, Salt Lake City, Utah, USA; Procter & Gamble Chemicals, Cincinnati, Ohio, USA.

Builders suitable for use in the present cleaning and/or treatment compositions can be obtained from Rhodia, Paris, France; Industrial Zeolite (UK) Ltd, Grays, Essex, UK; Koma, Nestemica, Czech Republic.

Polymers suitable for use in the present cleaning and/or treatment compositions can be obtained from BASF, Ludwigshafen, Germany, CP Kelco, Arnhem, Netherlands.

Photobleaches suitable for use in the present cleaning and/or treatment compositions can be obtained from Aldrich, Milwaukee, Wis., USA; Frontier Scientific, Logan, Utah, USA; Ciba Specialty Chemicals, Basel, Switzerland; BASF, Ludwigshafen, Germany; Lamberti S.p.A, Gallarate, Italy; Dayglo Color Corporation, Mumbai, India; Organic Dyestuffs Corp., East Providence, R.I., USA.

Hueing agents suitable for use in the present cleaning and/or treatment compositions can be obtained from Aldrich, Milwaukee, Wis., USA; Ciba Specialty Chemicals, Basel, Switzerland; BASF, Ludwigshafen, Germany; Dayglo Color Corporation, Mumbai, India; Organic Dyestuffs Corp., East Providence, R.I., USA; Dystar, Frankfurt, Germany; Lanxess, Leverkusen, Germany; Megazyme, Wicklow, Ireland; Clariant, Muttenz, Switzerland.

### Adjunct Materials

While not essential for the purposes of the present invention, the non-limiting list of adjuncts illustrated hereinafter are suitable for use in the instant compositions and may be desirably incorporated in certain embodiments of the invention, for example to assist or enhance cleaning performance, for treatment of the substrate to be cleaned, or to modify the aesthetics of the cleaning composition as is the case with perfumes, colorants, dyes or the like. Such adjunct are in addition to the materials already disclosed for use in the cleaning and/or treatment compositions described in the present specification. The precise nature of these additional components, and levels of incorporation thereof, will depend on the physical form of the composition and the nature of the cleaning operation for which it is to be used. Suitable adjunct materials include, but are not limited to, additional surfactants, additional builders, additional polymers, additional hueing agents, additional photobleaches, chelating agents, dye transfer inhibiting agents, dispersants, additional enzymes, and enzyme stabilizers, catalytic materials, bleach activators, hydrogen peroxide, sources of hydrogen peroxide, preformed peracids, polymeric dispersing agents, clay soil removal/anti-redeposition agents, brighteners, suds suppressors, dyes, perfumes, structure elasticizing agents, fabric softeners, carriers, hydrotropes, processing aids, solvents, additional hueing agents, structurants and/or pigments. In addition to the disclosure below, suitable examples of such other adjuncts and levels of use are found in U.S. Pat. Nos. 5,576,282, 6,306,812 B1 and 6,326,348 B1 that are incorporated by reference.

As stated, the adjunct ingredients are not essential to Applicants' compositions. Thus, certain embodiments of Applicants' compositions do not contain one or more of the following adjuncts materials: additional surfactants, additional builders, additional polymers, additional photobleaches, chelating agents, dye transfer inhibiting agents, dispersants, additional enzymes, and enzyme stabilizers, catalytic materials, bleach activators, hydrogen peroxide, sources of hydrogen peroxide, preformed peracids, polymeric dispersing agents, clay soil removal/anti-redeposition agents, brighteners, suds suppressors, dyes, perfumes, structure elasticizing agents, fabric softeners, carriers, hydrotropes, processing aids, solvents, additional hueing agents, structurants and/or pigments. However, when one or more adjuncts are present, such one or more adjuncts may be present as detailed below:

Bleaching Agents—The cleaning compositions of the present invention may comprise one or more bleaching agents. Suitable bleaching agents other than bleaching catalysts include photobleaches, bleach activators, hydrogen peroxide, sources of hydrogen peroxide, pre-formed peracids, bleach boosters and mixtures thereof. In general, when a bleaching agent is used, the compositions of the present invention may comprise from about 0.1% to about 50% or even from about 0.1% to about 25% bleaching agent by weight of the subject cleaning composition. Examples of suitable bleaching agents include:

(1) photobleaches. Suitable photobleaches being selected from the group consisting of xanthene dyes and mixtures thereof; sulfonated zinc phthalocyanine, sulfonated aluminium phthalocyanine, Eosin Y, Phoxine B, Rose Bengal, C.I. Food Red 14 and mixtures thereof; water soluble phthalocyanine;

(2) preformed peracids: Suitable preformed peracids include, but are not limited to, compounds selected from the group consisting of percarboxylic acids (for example phthalimidoperoxycaproic acid) and salts, percarbonic acids and salts, perimidic acids and salts, peroxymonosulfuric acids and salts, for example, Oxone®, and mixtures thereof. Suitable percarboxylic acids include hydrophobic and hydrophilic peracids having the formula R—(C═O)O—O-M wherein R is an alkyl group, optionally branched, having, when the peracid is hydrophobic, from 6 to 14 carbon atoms, or from 8 to 12 carbon atoms and, when the peracid is hydrophilic, less than 6 carbon atoms or even less than 4 carbon atoms; and M is a counter ion, for example, sodium, potassium or hydrogen;

(3) sources of hydrogen peroxide, for example, inorganic perhydrate salts, including alkali metal salts such as sodium salts of perborate (usually mono- or tetra-hydrate), percarbonate, persulphate, perphosphate, persilicate salts and mixtures thereof. In one aspect of the invention the inorganic perhydrate salts are selected from the group consisting of sodium salts of perborate, percarbonate and mixtures thereof. When employed, inorganic perhydrate salts are typically present in amounts of from 0.05 to 40 wt %, or 1 to 30 wt % of the overall composition and are typically incorporated into such compositions as a crystalline solid that may be coated. Suitable coatings include, inorganic salts such as alkali metal silicate, carbonate or borate salts or mixtures thereof, or organic materials such as water-soluble or dispersible polymers, waxes, oils or fatty soaps; and

(4) bleach activators having R—(C═O)-L wherein R is an alkyl group, optionally branched, having, when the bleach activator is hydrophobic, from 6 to 14 carbon atoms, or from 8 to 12 carbon atoms and, when the bleach activator is hydrophilic, less than 6 carbon atoms or even less than 4 carbon atoms; and L is leaving group. Examples of suitable leaving groups are benzoic acid and derivatives thereof—especially benzene sulphonate. Suitable bleach activators include dodecanoyl oxybenzene sulphonate, decanoyl oxybenzene sulphonate, decanoyl oxybenzoic acid or salts thereof, 3,5,5-trimethyl hexanoyloxybenzene sulphonate, tetraacetyl ethylene diamine (TAED) and nonanoyloxybenzene sulphonate (NOBS). Suitable bleach activators are also disclosed in WO 98/17767. While any suitable bleach activator may be employed, in one aspect of the invention the subject cleaning composition may comprise NOBS, TAED or mixtures thereof.

(5) Oxaziridinium-based bleach catalyst: A suitable oxaziridinium-based bleach catalyst has the formula:

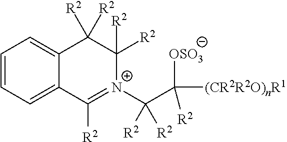

wherein: R¹ is selected from the group consisting of: H, a branched alkyl group containing from 3 to 24 carbons, and a linear alkyl group containing from 1 to 24 carbons; preferably, R¹ is a branched alkyl group comprising from 6 to 18 carbons, or a linear alkyl group comprising from 5 to 18 carbons, more preferably R¹ is selected from the group consisting of: 2-propylheptyl, 2-butyloctyl, 2-pentylnonyl, 2-hexyldecyl, n-hexyl, n-octyl, n-decyl, n-dodecyl, n-tetradecyl, n-hexadecyl, n-octadecyl, iso-nonyl, iso-decyl, iso-tridecyl and iso-pentadecyl; R² is independently selected from the group consisting of: H, a branched alkyl group comprising from 3 to 12 carbons, and a linear alkyl group comprising from 1 to 12 carbons; preferably R² is independently selected from H and methyl groups; and n is an integer from 0 to 1. In one aspect, such bleach booster may be selected from the group consisting of 2-[3-[(2-hexyldodecyl)oxy]-2-(sulfooxy)propyl]-3,4-dihydroisoquinolinium, inner salt; 3,4-dihydro-2-[3-[(2-pentylundecyl)oxy]-2-(sulfooxy)propyl]isoquinolinium, inner salt; 2-[3-[(2-butyldecyl)oxy]-2-(sulfooxy)propyl]-3,4-dihydroisoquinolinium, inner salt; 3,4-dihydro-2-[3-(octadecyloxy)-2-(sulfooxy)propyl]isoquinolinium, inner salt; 2-[3-(hexadecyloxy)-2-(sulfooxy)propyl]-3,4-dihydroisoquinolinium, inner salt; 3,4-dihydro-2-[2-(sulfooxy)-3-(tetradecyloxy)propyl]isoquinolinium, inner salt; 2-[3-(dodecyloxy)-2-(sulfooxy)propyl]-3,4-dihydroisoquinolinium, inner salt; 2-[3-[(3-hexyldecyl)oxy]-2-(sulfooxy)propyl]-3,4-dihydroisoquinolinium, inner salt; 3,4-dihydro-2-[3-[(2-pentylnonyl)oxy]-2-(sulfooxy)propyl]isoquinolinium, inner salt; 3,4-dihydro-2-[3-[(2-propylheptyl)oxy]-2-(sulfooxy)propyl]isoquinolinium, inner salt; 2-[3-[(2-butyloctyl)oxy]-2-(sulfooxy)propyl]-3,4-dihydroisoquinolinium, inner salt; 2-[3-(decyloxy)-2-(sulfooxy)propyl]-3,4-dihydroisoquinolinium, inner salt; 3,4-dihydro-2-[3-(octyloxy)-2-(sulfooxy)propyl]isoquinolinium, inner salt; 2-[3-[(2-ethylhexyl)oxy]-2-(sulfooxy)propyl]-3,4-dihydroisoquinolinium, inner salt and mixtures thereof.

As a practical matter, and not by way of limitation, the compositions and cleaning processes herein can be adjusted to provide on the order of at least 0.001 ppm of booster in the washing medium, from about 0.001 ppm to about 500 ppm, from about 0.005 ppm to about 150 ppm, or even from about 0.05 ppm to about 50 ppm, of booster in the wash liquor. In order to obtain such levels in the wash liquor, typical compositions herein will comprise from about 0.0002% to about 5%, from about 0.001% to about 1.5%, of booster, by weight of the cleaning compositions.

When present, the peracid and/or bleach activator is generally present in the composition in an amount of from about 0.1 to about 60 wt %, from about 0.5 to about 40 wt % or even from about 0.6 to about 10 wt % based on the composition. One or more hydrophobic peracids or precursors thereof may be used in combination with one or more hydrophilic peracid or precursor thereof.

The amounts of hydrogen peroxide source and peracid or bleach activator may be selected such that the molar ratio of available oxygen (from the peroxide source) to peracid is from 1:1 to 35:1, or even 2:1 to 10:1.

Surfactants—The cleaning compositions according to the present invention may comprise a surfactant or surfactant system wherein the surfactant can be selected from nonionic surfactants, anionic surfactants, cationic surfactants, ampholytic surfactants, zwitterionic surfactants, semi-polar nonionic surfactants and mixtures thereof. When present, surfactant is typically present at a level of from about 0.1% to about 60%, from about 1% to about 50% or even from about 5% to about 40% by weight of the subject composition.

Builders—The cleaning compositions of the present invention may comprise one or more detergent builders or builder systems. Builders include, but are not limited to, the alkali metal, ammonium and alkanolammonium salts of polyphosphates, alkali metal silicates, alkaline earth and alkali metal carbonates, aluminosilicate builders and polycarboxylate compounds, ether hydroxypolycarboxylates, copolymers of maleic anhydride with ethylene or vinyl methyl ether, 1,3,5-trihydroxy benzene-2,4,6-trisulphonic acid, and carboxymethyloxysuccinic acid, the various alkali metal, ammonium and substituted ammonium salts of polyacetic acids such as ethylenediamine tetraacetic acid and nitrilotriacetic acid, as well as polycarboxylates such as mellitic acid, succinic acid, citric acid, oxydisuccinic acid, polymaleic acid, benzene 1,3,5-tricarboxylic acid, carboxymethyloxysuccinic acid, and soluble salts thereof.

Chelating Agents—The cleaning compositions herein may contain a chelating agent. Suitable chelating agents include copper, iron and/or manganese chelating agents and mixtures thereof. When a chelating agent is used, the subject composition may comprise from about 0.005% to about 15% or even from about 3.0% to about 10% chelating agent by weight of the subject composition.

Dye Transfer Inhibiting Agents—The cleaning compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

Brighteners—The cleaning compositions of the present invention can also contain additional components that may tint articles being cleaned, such as fluorescent brighteners. Suitable fluorescent brightener levels include lower levels of from about 0.01, from about 0.05, from about 0.1 or even from about 0.2 wt % to upper levels of 0.5 or even 0.75 wt %.

Dispersants—The compositions of the present invention can also contain dispersants. Suitable water-soluble organic materials include the homo- or co-polymeric acids or their salts, in which the polycarboxylic acid comprises at least two carboxyl radicals separated from each other by not more than two carbon atoms.

Enzymes—The cleaning compositions can comprise one or more enzymes which provide cleaning performance and/or fabric care benefits. Examples of suitable enzymes include, but are not limited to, hemicellulases, peroxidases, proteases, cellulases, xylanases, lipases, phospholipases, esterases, cutinases, pectinases, mannanases, pectate lyases, keratinases, reductases, oxidases, phenoloxidases, lipoxygenases, ligninases, pullulanases, tannases, pentosanases, glucanases, arabinosidases, hyaluronidase, chondroitinase, laccase, amylases, or mixtures thereof. A typical combination is an enzyme cocktail that may comprise, for example, a protease and lipase in conjunction with amylase. When present in a cleaning composition, the aforementioned additional enzymes may be present at levels from about 0.00001% to about 2%, from about 0.0001% to about 1% or even from about 0.001% to about 0.5% enzyme protein by weight of the composition.

Enzyme Stabilizers—Enzymes for use in detergents can be stabilized by various techniques. The enzymes employed herein can be stabilized by the presence of water-soluble sources of calcium and/or magnesium ions in the finished compositions that provide such ions to the enzymes. In case of aqueous compositions comprising protease, a reversible protease inhibitor, such as a boron compound, for example, 4-formyl-phenylboronic acid can be added to further improve stability.

Catalytic Metal Complexes—Applicants' cleaning compositions may include catalytic metal complexes. One type of metal-containing bleach catalyst is a catalyst system comprising a transition metal cation of defined bleach catalytic activity, such as copper, iron, titanium, ruthenium, tungsten, molybdenum, or manganese cations, an auxiliary metal cation having little or no bleach catalytic activity, such as zinc or aluminum cations, and a sequestrate having defined stability constants for the catalytic and auxiliary metal cations, particularly ethylenediaminetetraacetic acid, ethylenediaminetetra(methylenephosphonic acid) and water-soluble salts thereof. Such catalysts are disclosed in U.S. Pat. No. 4,430,243.

If desired, the compositions herein can be catalyzed by means of a manganese compound. Such compounds and levels of use are well known in the art and include, for example, the manganese-based catalysts disclosed in U.S. Pat. No. 5,576,282.

Cobalt bleach catalysts useful herein are known, and are described, for example, in U.S. Pat. No. 5,597,936; U.S. Pat. No. 5,595,967. Such cobalt catalysts are readily prepared by known procedures, such as taught for example in U.S. Pat. No. 5,597,936, and U.S. Pat. No. 5,595,967.

Compositions herein may also suitably include a transition metal complex of ligands such as bispidones (WO 05/042532 A1) and/or macropolycyclic rigid ligands—abbreviated as “MRLs”. As a practical matter, and not by way of limitation, the compositions and processes herein can be adjusted to provide on the order of at least one part per hundred million of the active MRL species in the aqueous washing medium, and will typically provide from about 0.005 ppm to about 25 ppm, from about 0.05 ppm to about 10 ppm, or even from about 0.1 ppm to about 5 ppm, of the MRL in the wash liquor.

Suitable transition-metals in the instant transition-metal bleach catalyst include, for example, manganese, iron and chromium. Suitable MRLs include 5,12-diethyl-1,5,8,12-tetraazabicyclo[6.6.2]hexadecane.

Suitable transition metal MRLs are readily prepared by known procedures, such as taught for example in WO 00/32601, and U.S. Pat. No. 6,225,464.

Solvents—Suitable solvents include water and other solvents such as lipophilic fluids. Examples of suitable lipophilic fluids include siloxanes, other silicones, hydrocarbons, glycol ethers, glycerine derivatives such as glycerine ethers, perfluorinated amines, perfluorinated and hydrofluoroether solvents, low-volatility nonfluorinated organic solvents, diol solvents, other environmentally-friendly solvents and mixtures thereof.

### Processes of Making Compositions

The compositions of the present invention can be formulated into any suitable form and prepared by any process chosen by the formulator, non-limiting examples of which are described in Applicants' examples and in U.S. Pat. No. 4,990,280; U.S. 20030087791A1; U.S. 20030087790A1; and U.S. 20050003983A1.

### Method of Use

The present invention includes a method for cleaning a situs inter alia a surface or fabric. Such method includes the steps of contacting an embodiment of Applicants' cleaning composition, in neat form or diluted in a wash liquor, with at least a portion of a surface or fabric then optionally rinsing and optionally drying such surface or fabric. The surface or fabric may be subjected to a washing step prior to the aforementioned rinsing step. For purposes of the present invention, washing includes but is not limited to, scrubbing, and mechanical agitation. Drying of such surfaces or fabrics may be accomplished by any one of the common means employed either in domestic or industrial settings. Such means include but are not limited to forced air or still air drying at ambient or elevated temperatures at pressures between 5 and 0.01 atmospheres in the presence or absence of electromagnetic radiation, including sunlight, infrared, ultraviolet and microwave irradiation. In one aspect, said drying may be accomplished at temperatures above ambient by employing an iron wherein, for example, said fabric may be in direct contact with said iron for relatively short or even extended periods of time and wherein pressure may be exerted beyond that otherwise normally present due to gravitational force. In another aspect, said drying may be accomplished at temperatures above ambient by employing a dryer. Apparatus for drying fabric is well known and it is frequently referred to as a clothes dryer. In addition to clothes such appliances are used to dry many other items including towels, sheets, pillowcases, diapers and so forth and such equipment has been accepted as a standard convenience in many nations of the world substantially replacing the use of clothes lines for drying of fabric. Most dryers in use today use heated air which is passed over and or through the fabric as it is tumbled within the dryer. The air may be heated, for example, either electronically, via gas flame, or even with microwave radiation. Such air may be heated from about 15° C. to about 400° C., from about 25° C. to about 200° C., from about 35° C. to about 100° C., or even from about 40° C. to about 85° C. and used in the dryer to dry a surface and/or a fabric. Without being bound by theory, it is believed that additional bleaching may be obtained from organic catalyst remaining on the surface or fabric during and/or after drying thus it may be advantageous to dry said surface or fabric. As will be appreciated by one skilled in the art, the cleaning compositions of the present invention are ideally suited for use in laundry applications. Accordingly, the present invention includes a method for laundering a fabric. The method comprises the steps of contacting a fabric to be laundered with a said cleaning laundry solution comprising at least one embodiment of Applicants' cleaning composition, cleaning additive or mixture thereof. The fabric may comprise most any fabric capable of being laundered in normal consumer use conditions. The solution preferably has a pH of from about 8 to about 10.5. The compositions may be employed at concentrations of from about 500 ppm to about 15,000 ppm in solution. The water temperatures typically range from about 5° C. to about 90° C. The water to fabric ratio is typically from about 1:1 to about 30:1.

Thus, in one aspect, a method of treating and/or cleaning a surface or fabric comprising the steps of optionally washing and/or rinsing said surface or fabric, contacting said surface or fabric with any cleaning and/or treatment composition disclosed herein, then optionally washing and/or rinsing said surface and/or fabric then optionally letting drying said surface or fabric dry and/or actively drying said surface or fabric, is disclosed.

### EXAMPLES

Unless otherwise indicated, materials can be obtained from Aldrich, P.O. Box 2060, Milwaukee, Wis. 53201, USA.

### Example 1

Isolation of the fungal serine protease derived from Trichoderma reesei strain QM9414 defined by SEQ ID NO: 1

Full details of the protocol are given in D. Dienes et al, Enzyme and Microbial Technology 40 (2007) pp 1087. Briefly, a stock culture of parent Trichoderma reesei stain QM9414 is cultivated on 3.9% potato agar slants, in a modified minimal medium with glucose as sole carbon source and increased ammonium sulfate concentration in order to avoid exhaustion of nitrogen in the medium. The parent Trichoderma. reesei strain QM9414 is grown in 750 ml Erlenmeyer flasks in this modified medium. Erlenmeyer flasks containing 150 ml of the medium are inoculated with spores from 7-day-old culture. Fed-batch cultivations are carried out at 30° C. with shaking at 200 rpm. The glucose concentration is monitored daily and supplemented to 30-40 g/l. The pH is adjusted to 6.0 daily by the addition of 10% NaOH. After 8 days of cultivation the mycelia are removed by centrifugation and the supernatants are concentrated and then stored at −20° C. until analysis.

The 25 kDa protease is purified from 8 days culture filtrate by ion exchange chromatography and gel filtration. During anion exchange chromatography (pH 8) one peak is detected in the eluted fractions. The fractions showing activity on benzoyl-arginyl-p-nitroanilide are pooled, concentrated by ultrafiltration and subjected to size exclusion chromatography. About 95% purity is achieved with ion exchange separation and a subsequent gel filtration step.

### Examples 2-7

Granular laundry detergent compositions designed for hand washing or top-loading washing machines.

| | 2 | 3 | 4 | 5 | 6 | 7 |
|---|---|---|---|---|---|---|
| | (wt %) | (wt %) | (wt %) | (wt %) | (wt %) | (wt %) |
| Linear alkylbenzenesulfonate | 20 | 22 | 20 | 15 | 20 | 20 |
| C₁₂₋₁₄ Dimethylhydroxyethyl | 0.7 | 0.2 | 1 | 0.6 | 0.0 | 0 |
| ammonium chloride | | | | | | |
| AE3S | 0.9 | 1 | 0.9 | 0.0 | 0.5 | 0.9 |
| AE7 | 0.0 | 0.0 | 0.0 | 1 | 0.0 | 3 |
| Sodium tripolyphosphate | 5 | 0.0 | 4 | 9 | 2 | 0.0 |
| Zeolite A | 0.0 | 1 | 0.0 | 1 | 4 | 1 |
| 1.6R Silicate (SiO₂:Na₂O at | 7 | 5 | 2 | 3 | 3 | 5 |
| ratio 1.6:1) | | | | | | |
| Sodium carbonate | 25 | 20 | 25 | 17 | 18 | 19 |
| Polyacrylate MW 4500 | 1 | 0.6 | 1 | 1 | 1.5 | 1 |
| Random graft copolymer¹ | 0.1 | 0.2 | 0.0 | 0.0 | 0.0 | 0.0 |
| Carboxymethyl cellulose | 1 | 0.3 | 1 | 1 | 1 | 1 |
| Stainzyme ® (20 mg active/g) | 0.1 | 0.2 | 0.1 | 0.2 | 0.1 | 0.1 |
| Bacterial protease (Savinase ®, | 0.1 | 0.1 | 0.1 | 0.1 | | 0.1 |
| 32.89 mg active/g) | | | | | | |
| Natalase ® (8.65 mg active/g) | 0.1 | 0.0 | 0.1 | 0.0 | 0.1 | 0.1 |
| Lipex ® (18 mg active/g) | 0.03 | 0.07 | 0.3 | 0.1 | 0.07 | 0.4 |
| Fungal protease of the present | 0.1 | 0.2 | 0.2 | 0.2 | 0.1 | 0.4 |
| invention (20 mg active/g) | | | | | | |
| Fluorescent Brightener 1 | 0.06 | 0.0 | 0.06 | 0.18 | 0.06 | 0.06 |
| Fluorescent Brightener 2 | 0.1 | 0.06 | 0.1 | 0.0 | 0.1 | 0.1 |
| DTPA | 0.6 | 0.8 | 0.6 | 0.25 | 0.6 | 0.6 |
| MgSO₄ | 1 | 1 | 1 | 0.5 | 1 | 1 |
| Sodium Percarbonate | 0.0 | 5.2 | 0.1 | 0.0 | 0.0 | 0.0 |
| Sodium Perborate | 4.4 | 0.0 | 3.85 | 2.09 | 0.78 | 3.63 |
| Monohydrate | | | | | | |
| NOBS | 1.9 | 0.0 | 1.66 | 0.0 | 0.33 | 0.75 |
| TAED | 0.58 | 1.2 | 0.51 | 0.0 | 0.015 | 0.28 |
| Sulphonated zinc | 0.0030 | 0.0 | 0.0012 | 0.0030 | 0.0021 | 0.0 |
| phthalocyanine | | | | | | |
| S-ACMC | 0.1 | 0.0 | 0.0 | 0.0 | 0.06 | 0.0 |
| Direct Violet 9 | 0.0 | 0.0 | 0.0003 | 0.0005 | 0.0003 | 0.0 |
| Acid Blue 29 | 0.0 | 0.0 | 0.0 | 0.0 | 0.0 | 0.0003 |

| | |
|---|---|
| Sulfate/Moisture | Balance |

### Examples 8-13

Granular laundry detergent compositions designed for front-loading automatic washing machines.

| | 8 | 9 | 10 | 11 | 12 | 13 |
|---|---|---|---|---|---|---|
| | (wt %) | (wt %) | (wt %) | (wt %) | (wt %) | (wt %) |
| Linear alkylbenzenesulfonate | 8 | 7.1 | 7 | 6.5 | 7.5 | 7.5 |
| AE3S | 0 | 4.8 | 0 | 5.2 | 4 | 4 |
| C12-14 Alkylsulfate | 1 | 0 | 1 | 0 | 0 | 0 |
| AE7 | 2.2 | 0 | 3.2 | 0 | 0 | 0 |
| C₁₀₋₁₂ Dimethyl | 0.75 | 0.94 | 0.98 | 0.98 | 0 | 0 |
| hydroxyethylammonium chloride | | | | | | |
| Crystalline layered silicate (δ- | 4.1 | 0 | 4.8 | 0 | 0 | 0 |
| Na₂Si₂O₅) | | | | | | |
| Zeolite A | 5 | 0 | 5 | 0 | 2 | 2 |
| Citric Acid | 3 | 5 | 3 | 4 | 2.5 | 3 |
| Sodium Carbonate | 15 | 20 | 14 | 20 | 23 | 23 |
| Silicate 2R (SiO₂:Na₂O at ratio | 0.08 | 0 | 0.11 | 0 | 0 | 0 |
| 2:1) | | | | | | |
| Soil release agent | 0.75 | 0.72 | 0.71 | 0.72 | 0 | 0 |
| Acrylic Acid/Maleic Acid | 1.1 | 3.7 | 1.0 | 3.7 | 2.6 | 3.8 |
| Copolymer | | | | | | |
| Carboxymethylcellulose | 0.15 | 1.4 | 0.2 | 1.4 | 1 | 0.5 |
| Bacterial protease (84 mg | 0.2 | 0.2 | 0.3 | 0.15 | 0.12 | 0.13 |
| active/g) | | | | | | |
| Stainzyme ® (20 mg active/g) | 0.2 | 0.15 | 0.2 | 0.3 | 0.15 | 0.15 |
| Lipex ® (18.00 mg active/g) | 0.05 | 0.15 | 0.1 | 0 | 0 | 0 |
| Natalase ® (8.65 mg active/g) | 0.1 | 0.2 | 0 | 0 | 0.15 | 0.15 |
| Celluclean ™ (15.6 mg active/g) | 0 | 0 | 0 | 0 | 0.1 | 0.1 |
| Fungal protease of the present | 0.2 | 0.1 | 0.2 | 0.2 | 0.2 | 0.2 |
| invention (20 mg active/g) | | | | | | |
| TAED | 3.6 | 4.0 | 3.6 | 4.0 | 2.2 | 1.4 |
| Percarbonate | 13 | 13.2 | 13 | 13.2 | 16 | 14 |
| Na salt of Ethylenediamine-N,N′- | 0.2 | 0.2 | 0.2 | 0.2 | 0.2 | 0.2 |
| disuccinic acid, (S,S) isomer | | | | | | |
| (EDDS) | | | | | | |
| Hydroxyethane di phosphonate | 0.2 | 0.2 | 0.2 | 0.2 | 0.2 | 0.2 |
| (HEDP) | | | | | | |
| MgSO₄ | 0.42 | 0.42 | 0.42 | 0.42 | 0.4 | 0.4 |
| Perfume | 0.5 | 0.6 | 0.5 | 0.6 | 0.6 | 0.6 |
| Suds suppressor agglomerate | 0.05 | 0.1 | 0.05 | 0.1 | 0.06 | 0.05 |
| Soap | 0.45 | 0.45 | 0.45 | 0.45 | 0 | 0 |
| Sulphonated zinc phthalocyanine | 0.0007 | 0.0012 | 0.0007 | 0 | 0 | 0 |
| (active) | | | | | | |
| S-ACMC | 0.01 | 0.01 | 0 | 0.01 | 0 | 0 |
| Direct Violet 9 (active) | 0 | 0 | 0.0001 | 0.0001 | 0 | 0 |

| | |
|---|---|
| Sulfate/Water & Miscellaneous | Balance |

Any of the above compositions is used to launder fabrics at a concentration of 7000 to 10000 ppm in water, 20-90° C., and a 5:1 water:cloth ratio. The typical pH is about 10. The fabrics are then dried. In one aspect, the fabrics are actively dried using a dryer. In one aspect, the fabrics are actively dried using an iron. In another aspect, the fabrics are merely allowed to dry on a line wherein they are exposed to air and optionally sunlight.

### Examples 14-19 Heavy Duty Liquid Laundry Detergent Compositions

| | 14 | 15 | 16 | | | |
|---|---|---|---|---|---|---|
| | (wt %) | (wt %) | (wt %) | 17 (wt %) | 18 (wt %) | 19 (wt %) |
| AES C₁₂₋₁₅ alkyl | 11 | 10 | 4 | 6.32 | 0 | 0 |
| ethoxy (1.8) sulfate | | | | | | |
| AE3S | 0 | 0 | 0 | 0 | 2.4 | 0 |
| Linear alkyl | 1.4 | 4 | 8 | 3.3 | 5 | 8 |
| benzene sulfonate | | | | | | |
| HSAS | 3 | 5.1 | 3 | 0 | 0 | 0 |
| Sodium formate | 1.6 | 0.09 | 1.2 | 0.04 | 1.6 | 1.2 |
| Sodium hydroxide | 2.3 | 3.8 | 1.7 | 1.9 | 1.7 | 2.5 |
| Monoethanolamine | 1.4 | 1.49 | 1.0 | 0.7 | 0 | 0 |
| Diethylene glycol | 5.5 | 0.0 | 4.1 | 0.0 | 0 | 0 |
| AE9 | 0.4 | 0.6 | 0.3 | 0.3 | 0 | 0 |
| AE7 | 0 | 0 | 0 | 0 | 2.4 | 6 |
| Chelant | 0.15 | 0.15 | 0.11 | 0.07 | 0.5 | 0.11 |
| Citric Acid | 2.5 | 3.96 | 1.88 | 1.98 | 0.9 | 2.5 |
| C₁₂₋₁₄ dimethyl | 0.3 | 0.73 | 0.23 | 0.37 | 0 | 0 |
| Amine Oxide | | | | | | |
| C₁₂₋₁₈ Fatty Acid | 0.8 | 1.9 | 0.6 | 0.99 | 1.2 | 0 |
| 4-formyl- | 0 | 0 | 0 | 0 | 0.05 | 0.02 |
| phenylboronic acid | | | | | | |
| Borax | 1.43 | 1.5 | 1.1 | 0.75 | 0 | 1.07 |
| Ethanol | 1.54 | 1.77 | 1.15 | 0.89 | 0 | 3 |
| Ethoxylated (EO₁₅) | 0.3 | 0.33 | 0.23 | 0.17 | 0.0 | 0.0 |
| tetraethylene | | | | | | |
| pentamine | | | | | | |
| Ethoxylated | 0.8 | 0.81 | 0.6 | 0.4 | 1 | 1 |
| hexamethylene | | | | | | |
| diamine | | | | | | |
| 1,2-Propanediol | 0.0 | 6.6 | 0.0 | 3.3 | 0.5 | 2 |
| Bacterial protease | 0.8 | 0.6 | 0.7 | 0.9 | 0.7 | 0.6 |
| (40.6 mg active/g) | | | | | | |
| Mannaway ® (25 mg | 0.07 | 0.05 | 0.045 | 0.06 | 0.04 | 0.045 |
| active/g) | | | | | | |
| Stainzyme ® (15 mg | 0.3 | 0.2 | 0.3 | 0.1 | 0.2 | 0.4 |
| active/g) | | | | | | |
| Natalase ® (29 mg | 0 | 0.2 | 0.1 | 0.15 | 0.07 | 0 |
| active/g) | | | | | | |
| Lipex ® (18 mg | 0.4 | 0.2 | 0.3 | 0.1 | 0.2 | 0 |
| active/g) | | | | | | |
| Fungal protease of | 0.2 | 0.1 | 0.2 | 0.2 | 0.1 | 0.1 |
| the present | | | | | | |
| invention (20 mg | | | | | | |
| active/g) | | | | | | |
| Liquitint ® Violet | 0.006 | 0.002 | 0 | 0 | 0 | 0.002 |
| CT (active) | | | | | | |
| S-ACMC | — | — | 0.01 | 0.05 | 0.01 | 0.02 |

| | |
|---|---|
| Water, perfume, | Balance |
| dyes & other | |
| components | |

### Example 20

| | 19 (wt %) |
|---|---|
| Alkylbenzene sulfonic acid | 21.0 |
| C₁₄₋₁₅ alkyl 8-ethoxylate | 18.0 |
| C₁₂₋₁₈ Fatty acid | 15.0 |
| Bacterial protease (40.6 mg active/g) | 1.5 |
| Natalase ® (29 mg active/g) | 0.2 |
| Mannanase (Mannaway ®, 11 mg active/g) | 0.1 |
| Xyloglucanase (Whitezyme ®, 20 mg active/g) | 0.2 |
| Fungal protease of the present invention (20 mg | 0.2 |
| active/g) | |
| A compound having the following general | 2.0 |
| structure: bis((C₂H₅O)(C₂H₄O)n)(CH₃)—N⁺— | |
| CₓH₂ₓ—N⁺—(CH₃)-bis((C₂H₅O)(C₂H₄O)n), | |
| wherein n = from 20 to 30, and x = from 3 to 8, | |
| or sulphated or sulphonated variants thereof | |
| Ethoxylated Polyethylenimine ² | 0.8 |
| Hydroxyethane diphosphonate (HEDP) | 0.8 |
| Fluorescent Brightener 1 | 0.2 |
| Solvents (1,2 propanediol, ethanol), stabilizers | 15.0 |
| Hydrogenated castor oil derivative structurant | 0.1 |
| Perfume | 1.6 |
| Core Shell Melamine-formaldehyde encapsulate | 0.10 |
| of perfume | |
| Ethoxylated thiophene Hueing Dye | 0.004 |
| Buffers (sodium hydroxide, | To pH 8.2 |
| Monoethanolamine) | |
| Water* and minors (antifoam, aesthetics) | To 100% |

*Based on total cleaning and/or treatment composition weight, a total of no more than 7% water
¹ Random graft copolymer is a polyvinyl acetate grafted polyethylene oxide copolymer having a polyethylene oxide backbone and multiple polyvinyl acetate side chains. The molecular weight of the polyethylene oxide backbone is about 6000 and the weight ratio of the polyethylene oxide to polyvinyl acetate is about 40 to 60 and no more than 1 grafting point per 50 ethylene oxide units.
² Polyethyleneimine (MW = 600) with 20 ethoxylate groups per —NH.
*Remark: all enzyme levels expressed as % enzyme raw material

### Raw Materials and Notes for Composition Examples 1-20

Linear alkylbenzenesulfonate having an average aliphatic carbon chain length C₁₁-C₁₂ supplied by Stepan, Northfield, Ill., USA

C₁₂₋₁₄ Dimethylhydroxyethyl ammonium chloride, supplied by Clariant GmbH, Sulzbach, Germany

AE3S is C₁₂₋₁₅ alkyl ethoxy (3) sulfate supplied by Stepan, Northfield, Ill., USA

AE7 is C₁₂₋₁₅ alcohol ethoxylate, with an average degree of ethoxylation of 7, supplied by Huntsman, Salt Lake City, Utah, USA

AE9 is C₁₂₋₁₃ alcohol ethoxylate, with an average degree of ethoxylation of 9, supplied by Huntsman, Salt Lake City, Utah, USA HSAS is a mid-branched primary alkyl sulfate with carbon chain length of about 16-17

Sodium tripolyphosphate is supplied by Rhodia, Paris, France

Zeolite A is supplied by Industrial Zeolite (UK) Ltd, Grays, Essex, UK

1.6R Silicate is supplied by Koma, Nestemica, Czech Republic

Sodium Carbonate is supplied by Solvay, Houston, Tex., USA

Polyacrylate MW 4500 is supplied by BASF, Ludwigshafen, Germany

Carboxymethyl cellulose is Finnfix® V supplied by CP Kelco, Arnhem, Netherlands

Suitable chelants are, for example, diethylenetetraamine pentaacetic acid (DTPA) supplied by Dow Chemical, Midland, Mich., USA or Hydroxyethane di phosphonate (HEDP) supplied by Solutia, St Louis, Mo., USA Bagsvaerd, Denmark

Savinase®, Natalase®, Stainzyme®, Lipex®, Celluclean™, Mannaway® and Whitezyme® are all products of Novozymes, Bagsvaerd, Denmark.

Bacterial protease (examples 8-13) described in U.S. Pat. No. 6,312,936 B1 supplied by Genencor International, Palo Alto, Calif., USA

Bacterial protease (examples 14-20) described in U.S. Pat. No. 4,760,025 is supplied by Genencor International, Palo Alto, Calif., USA

Fluorescent Brightener 1 is Tinopal® AMS, Fluorescent Brightener 2 is Tinopal® CBS-X, Sulphonated zinc phthalocyanine and Direct Violet 9 is Pergasol® Violet BN-Z all supplied by Ciba Specialty Chemicals, Basel, Switzerland

Sodium percarbonate supplied by Solvay, Houston, Tex., USA

Sodium perborate is supplied by Degussa, Hanau, Germany

NOBS is sodium nonanoyloxybenzenesulfonate, supplied by Future Fuels, Batesville, Ark., USA

TAED is tetraacetylethylenediamine, supplied under the Peractive® brand name by Clariant GmbH, Sulzbach, Germany

S-ACMC is carboxymethylcellulose conjugated with C.I. Reactive Blue 19, sold by Megazyme, Wicklow, Ireland under the product name AZO-CM-CELLULOSE, product code S-ACMC.

Soil release agent is Repel-o-Tex® PF, supplied by Rhodia, Paris, France

Acrylic Acid/Maleic Acid Copolymer is molecular weight 70,000 and acrylate:maleate ratio 70:30, supplied by BASF, Ludwigshafen, Germany

Na salt of Ethylenediamine-N,N′-disuccinic acid, (S,S) isomer (EDDS) is supplied by Octel, Ellesmere Port, UK

Hydroxyethane di phosphonate (HEDP) is supplied by Dow Chemical, Midland, Mich., USA

Suds suppressor agglomerate is supplied by Dow Corning, Midland, Mich., USA

HSAS is mid-branched alkyl sulfate as disclosed in U.S. Pat. No. 6,020,303 and U.S. Pat. No. 6,060,443

C₁₂₋₁₄ dimethyl Amine Oxide is supplied by Procter & Gamble Chemicals, Cincinnati, Ohio, USA

Liquitint® Violet CT is supplied by Milliken, Spartanburg, S.C., USA

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A cleaning composition comprising a fungal serine protease having at least 95% identity to SEQ ID NO: 6 and an additional cleaning material.

2. The cleaning composition of claim 1, wherein said additional cleaning material is selected from the group consisting of lipase, hueing dye, bacterial protease, bacterial amylase, endo-beta-1,4-glucanase, perhydrolase, perfume microcapsule, carboxymethylcellulose, bleach catalyst, and mixtures thereof.

3. The cleaning composition of claim 2, wherein said:
a) lipase is selected from the group consisting of variants of the Humicola lanuginosa lipase comprising a substitution of an electrically neutral or negatively charged amino acid with R or K at any of positions 3, 224, 229, 231 and 233, in one aspect, a variant comprising T231R and N233R mutations;
b) hueing dye is selected from the group consisting of direct violet 7, direct violet 9, direct violet 11, direct violet 26, direct violet 31, direct violet 35, direct violet 40, direct violet 41, direct violet 51, direct violet 66, direct violet 99, acid violet 50, acid blue 9, acid violet 17, acid black 1, acid red 17, acid blue 29, solvent violet 13, disperse violet 27 disperse violet 26, disperse violet 28, disperse violet 63 and disperse violet 77, basic blue 16, basic blue 65, basic blue 66, basic blue 67, basic blue 71, basic blue 159, basic violet 19, basic violet 35, basic violet 38, basic violet 48; basic blue 3, basic blue 75, basic blue 95, basic blue 122, basic blue 124, basic blue 141, thiazolium dyes, reactive blue 19, reactive blue 163, reactive blue 182, reactive blue 96, and polymeric dyes;
c) bacterial protease is selected from the group consisting of wild-type and variants of subtilisins derived from Bacillus lentus, B. alkalophilus, B. subtilis, and B. amyloliquefaciens.
d) bacterial amylase is selected from the group consisting of wild-type and variants of amylase AA560 from Bacillus sp. DSM 12649, and wild-type and variants of amylase SP722 from Bacillus sp. NCIB 12513.
e) endo-beta-1,4-glucanase is selected from the group consisting of wild-type and variants of the 20 kDa endoglucanase from Melanocarpus albomyces, wild-type and variants of the endoglucanase from Bacillus sp. AA349; and wild-type and variants of the XYG1006 endoglucanase from Paenibacillus polymyxa
f) perhydrolase is selected from the group consisting of variants of the Mycobacterium smegmatis perhydrolase, and variants of the CE-7 perhydrolases;
g) perfume microcapsule is selected from the group consisting of core/shell perfume microcapsules, in one aspect comprising a melamine/formaldehyde resin shell;
h) carboxymethylcellulose is selected from the group consisting of carboxymethycellulose derivatives having a degree of carboxymethyl substitution of from about 0.5 to about 0.95; and
i) bleaching material selected from the group consisting of catalytic metal complexes, photobleaches, bleach activators, hydrogen peroxide, sources of hydrogen peroxide, pre-formed peracids, bleach boosters and mixtures thereof.

4. The cleaning composition of claim 1, said cleaning composition comprising, based on total composition weight, from about 0.00001% to about 2% of said fungal serine protease.

5. The cleaning composition of claim 1, wherein said additional cleaning material is selected from the group consisting of surfactants, chelating agents, dye transfer inhibiting agents, dispersants, additional enzymes, and enzyme stabilizers, catalytic materials, bleaching agents, polymeric dispersing agents, clay soil removal/anti-redeposition agents, brighteners, suds suppressors, dyes, perfumes, perfume microcapsules, structure elasticizing agents, fabric softeners, carriers, hydrotropes, processing aids, solvents, pigments, hueing agents, photobleaches, structurants, and mixtures thereof.

6. The cleaning composition of claim 1, said cleaning composition comprising an additional enzyme.

7. The cleaning composition of claim 6, wherein said additional enzyme is selected from the group consisting of hemicellulases, peroxidases, proteases, cellulases, xylanases, lipases, phospholipases, perhydrolases, esterases, cutinases, pectinases, mannanases, pectate lyases, keratinases, reductases, oxidoreductases, phenoloxidases, lipoxygenases, ligninases, pullulanases, tannases, pentosanases, glucanases, arabinosidases, hyaluronidase, chondroitinase, laccase, amylases, and mixtures thereof.

8. The cleaning composition of claim 7, wherein said additional enzyme is selected from the group consisting of:
a.) first cycle lipases;
b.) cutinases;
c.) alpha-amylases;
d.) bacterial proteases;
e.) microbial-derived endoglucanases; and
f.) mixtures thereof.

9. The cleaning composition of claim 5, said cleaning composition comprising a surfactant selected from the group consisting of:
a.) anionic surfactants selected from the group consisting of linear alkylbenzene-sulfonate (LAS), alcohol ethoxysulfate (AES), mid-branched alkyl sulfates (HSAS) and mixtures thereof;
b.) non ionic alcohol ethoxylates,
c.) amine oxides; and
d.) mixtures thereof.

10. The cleaning composition of claim 5, said cleaning composition comprising a polymeric dispersing agents selected from the group consisting of
a.) polyacrylates;
b.) maleic/acrylic acid copolymers;
c.) cellulose-derived polymers;
d.) polyethyleneimine polymer; and
e.) mixtures thereof.

11. The cleaning composition of claim 5, said cleaning composition comprising a fabric hueing agent selected from the group consisting of
a.) dyes;
b.) dye-clay conjugates comprising at least one cationic/basic dye and a smectite clay; and
c.) mixtures thereof.

12. The cleaning composition of claim 11, said cleaning composition comprising, based on total product weight, from about 0.00003% to about 0.3% hueing agent.

13. The cleaning composition of claim 1, said cleaning composition comprising, based on total product weight, less than 15% builder.

14. The cleaning composition of claim 1 wherein said cleaning composition is a multi-compartment unit dose.

15. The cleaning composition of claim 1 wherein said cleaning composition is a multi-compartment unit dose, wherein the fungal serine protease is in a different compartment to any additional enzymes and/or chelant.

16. The cleaning composition of claim 1, wherein said cleaning composition is a hand dishwashing or machine dishwashing composition.

17. The cleaning composition of claim 1, wherein said cleaning composition comprises, based on total cleaning composition weight, a total of no more than 20% water.

18. The cleaning composition of claim 1, wherein said cleaning composition comprises based on total cleaning composition weight, from about 10% to about 70% of a water-miscible organic solvent having a molecular weight of greater than 70 Daltons.

19. The cleaning composition of claim 1, said cleaning composition comprising a perfume microcapsule comprising a core and a shell that encapsulates said core, said perfume microcapsule having a D [4,3] average particle of from about 0.01 microns to about 200 microns.
